(19) United States
(12) Patent Application Publication
(10) Pub. No.: US 2022/0072043 A1
(43) Pub. Date: Mar. 10, 2022

(54) COMBINATION THERAPY COMPRISING CANCER-TARGETED CAR-T CELLS AND A METHOD OF USING SAME FOR A TREATMENT FOR CANCER

(71) Applicants: Mustang Bio, Inc, Worchester, MA (US); Beckman Research Institute of the City of Hope, Duarte, CA (US)

(72) Inventors: Knut Niss, Worchester, MA (US); Sadik H. Kassim, Milton, MA (US); Christine E. Brown, Duarte, CA (US); Stephen J. Forman, Duarte, CA (US); Behnam Badie, Duarte, CA (US)

(73) Assignees: Mustang Bio, Inc., Worchester, MA (US); Beckman Research Institute of the City of Hope, Duarte, CA (US)

(21) Appl. No.: 17/470,843

(22) Filed: Sep. 9, 2021

Related U.S. Application Data
(60) Provisional application No. 63/076,805, filed on Sep. 10, 2020.

(51) Int. Cl. A61K 35/17; C07K 14/725; C07K 14/705; C07K 14/73; C07K 14/71; C07K 16/28; C07K 16/32; A61K 35/763

(52) U.S. Cl. CPC A61K 35/17; C07K 14/7051; C07K 14/70517; C07K 14/70514; C07K 14/70578; A61K 38/00; C07K 16/2866; C07K 16/32; A61K 35/763; C07K 2319/03; C07K 14/71

(57) ABSTRACT

Described are improved methods for treating solid tumors, such as malignant gliomas, with a dual approach utilizing a CAR-T immunotherapy and a viral oncolytic therapy.

### CROSS REFERENCE TO RELATED APPLICATION

This application claims priority under 35 U.S.C. § 119(e) to U.S. Provisional Patent Application No. 63/076,805, filed Sep. 10, 2020, the entire contents of which are incorporated herein by reference.

### FIELD OF INVENTION

The present disclosure relates to the field of tumor immunotherapy, in particular to a malignant glioma CAR-T therapy.

### STATEMENT REGARDING SEQUENCE LISTING

The sequence listing associated with this application is provided in text format in lieu of a paper copy, and is hereby incorporated by reference into the specification. The name of the text file containing the sequence listing is 118180-0600_ST25.txt. the text file is ˜28 kb, was created on Sep. 8, 2020, and is being submitted electronically via EFS-Web.

### BACKGROUND

Novel treatments using T cells engineered to express immune receptors have resulted in promising immunotherapies for a wide swath of intractable diseases, including cancer and autoimmune diseases.

Cancer is a remarkable threat to human health, leading to reduced quality of life and, too often, death. The burden placed on national, regional and local healthcare organizations to treat and prevent the various forms of cancer is significant in terms of the resources and manpower required. One of the primary weapons humans have to combat disease is a functioning immune system. Antigens, including mutant antigens, are powerful targets for tumor destruction, e.g., in mice, and tumor-infiltrating lymphocytes targeting these antigens can cause durable tumor regression in patients.

The high affinity interleukin-13 receptor α2 (IL13Rα2) is selectively expressed at a high frequency by glioblastoma multiforme (GBM), and also in other tumor types. One approach for targeting this tumor-specific receptor utilizes the cognate ligand, IL-13, conjugated to cytotoxic molecules. This approach; however, lacks specificity because the lower affinity receptor for IL-13, IL13Rα1, is widely expressed in non-cancer tissues. Human epidermal growth factor receptor 2 (HER2) is a validated cancer target in several types of cancer, most notably breast cancer, but it has recently been determined that HER is also express in up to 80% of GBMs but not by normal postnatal neurons or glia.

Many human cancers lack specific antigens that are predictably present on or in cancerous tissues and are capable as serving as effective mitigation targets by immune cells such as T cells. Every cancer cell type harbors a unique set of mutations causing different tumor-specific antigens. Identifying an effective unique antigen and isolating an appropriate T cell receptor for transduction of autologous T cells for adoptive immunotherapy continues to be difficult despite advances in the state of the art. The success of utilizing adoptive T cell immunotherapies with chimeric antigen receptors (CARs) and bispecific T cell engaging proteins (BiTEs) is; however, largely restricted to neoplastic hematological disorders. A number of factors that make adoptive immunotherapy difficult in treating solid tumors, largely attributed to an unfavorable tumor microenvironment to T cells.

Thus, there is a need for identifying adoptive immunotherapies for treating solid tumors, particularly GBM. The present disclosure addresses this need.

### SUMMARY OF THE DISCLOSURE

In some aspects, the present disclosure is generally drawn to a method of treating glioblastoma multiforme (GBM) in a subject comprising, administering to a subject with GBM (i) a population of cells that express a chimeric antigen receptor (CAR), and (ii) an oncolytic virus. In some aspects, the population of cells that express the CAR and the oncolytic virus are administered concurrently. In some aspects, the population of cells that express the CAR and the oncolytic virus are administered sequentially. In some aspects, the population of cells that express the CAR are administered first and the oncolytic virus is administered second. In some aspects, the oncolytic virus is administered first and the population of cells that express the CAR are administered second. In some aspects, the subject has previously undergone surgery to remove a primary GBM tumor and/or a metastatic tumor.

In some aspects, the population of cells that express that CAR comprise T cells. In some aspects, the T cells are central memory T cells. In some aspects, the population of cells that express that CAR comprise NK cells.

In some aspects, the CAR comprises a binding domain, a spacer domain, a transmembrane domain, a costimulatory domain, and a CD3ζ signaling domain. In some aspects, the CAR selectively binds to IL-13Rα2. In some aspects, the binding domain comprises SEQ ID NO:1. In some aspects, the spacer domain comprises an IgG4 Fc domain. In some aspects, the IgG4 Fc domain comprises SEQ ID NO:2, SEQ ID NO:3, SEQ ID NO:4, or SEQ ID NO:5.

In some aspects, the transmembrane domain comprises a CD4 transmembrane domain. In some aspects, the CD4 transmembrane domain comprises MALIVLGGVAGLLLFIGLGIFF (SEQ ID NO: 6). In some aspects, the transmembrane domain comprises a CD8 transmembrane domain. In some aspects, or the CD8 transmembrane domain comprises IYIWAPLAGTCGVLLLSLVIT (SEQ ID NO: 11), IYIWAPLAGTCGVLLLSLVITLY (SEQ ID NO: 12), or IYIWAPLAGTCGVLLLSLVITLYC (SEQ ID NO: 13). In some aspects, the costimulatory domain comprises a 4-1BB costimulatory domain. In some aspects, the 4-1BB costimulatory domain comprises SEQ ID NO:7. In some aspects, the CAR comprises SEQ ID NO:8 or SEQ ID NO: 14.

In some aspects, the population of cells that express the CAR also express a selection tag. In some aspects, the selection tag is a truncated CD19 sequence (CD19t) or a truncated epidermal growth factor receptor sequence (EGFRt). In some aspects, the CD19t or EGFRt sequence is separated from the CAR by a T2A spacer sequence.

In some aspects, the oncolytic virus is a genetically engineered herpes simplex virus type 1 (HSV-1). In some aspects, the oncolytic virus is replication-competent. In some aspects, a ICP34.5 gene is deleted from the genome of the oncolytic virus and a gene encoding human cytomegalovirus (HCMV) IRS1 gene is introduced into the genome of the oncolytic virus. In some aspects, the oncolytic virus is C134.

In some aspects, the population of cells that express the CAR are central memory T cells and the CAR comprises SEQ ID NO: 8 or SEQ ID NO: 14, and wherein the oncolytic virus is C134. In some aspects, the population of cells expressing the CAR and the oncolytic virus are administered via the same route of administration. In some aspects, the population of cells expressing the CAR and the oncolytic virus are administered via different routes of administration.

In some aspects, the route of administration is selected from the group consisting of intraventricular, intratumoral, intrathecal, and into a resected tumor cavity. In some aspects, the population of cells expressing the CAR and the oncolytic virus are administered directly into the central nervous system (CNS) of the subject.

The inventions described and claimed herein have many attributes and embodiments including, but not limited to, those set forth or described or referenced in this Brief Summary and the following Brief Description of the Drawings and Detailed Description. It is not intended to be all-inclusive and the inventions described and claimed herein are not limited to or by the features or embodiments identified in this Brief Summary, which is included for purposes of illustration only and not restriction. Additional embodiments may be disclosed in the Detailed Description below.

### BRIEF DESCRIPTION OF THE FIGURES

FIG. 1 depicts a graphical representation of an exemplary scheme of administrations and assays performed in Example 4.

### DETAILED DESCRIPTION

The disclosure is drawn to methods of treating a wide range of cancers characterized by cells expressing or presenting IL-13Rα2 or HER2 by administering a combination of (1) an immunological cell expressing a chimeric antigen receptor (CAR) that specifically interacts (e.g., binds to) with IL-13Rα2 or HER2, and (2) an oncolytic virus.

### I. Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, the terms “about” or “approximately” when preceding a numerical value indicates the value plus or minus a range of 10% of the value. For example “about 10” should be understood as “10” and “9-11.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various aspects of the disclosure can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, a “control” is an alternative sample used in an experiment for comparison purpose. A control can be “positive” or “negative.” A “control sample” or “reference sample” as used herein, refer to a sample or reference that acts as a control for comparison to an experimental sample. For example, an experimental sample comprises compound A, B, and C in a vial, and the control may be the same type of sample treated identically to the experimental sample, but lacking one or more of compounds A, B, or C.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. Polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “composition” is intended to encompass a product containing the specified ingredients (e.g., an oncolytic virus as provided herein) in, optionally, the specified amounts, as well as any product which results, directly or indirectly, from combination of the specified ingredients in, optionally, the specified amounts.

As used herein, the term “vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai virus vectors, adenovirus vectors, adeno-associated virus vectors, HSV vectors, retrovirus vectors, lentivirus vectors, and the like.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “expression” is defined as the transcription and/or translation of a particular nucleotide sequence driven by its promoter.

As used herein, the term “epitope” is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide of the present invention can be an epitope.

Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns to the extent that the nucleotide sequence encoding the protein may in some version contain an intron(s).

As used herein, the term “autologous” is meant to refer to any material derived from the same individual to which it is later to be re-introduced into the individual.

As used herein, “Allogeneic” refers to any material derived from a different animal of the same species. “Xenogeneic” refers to any material derived from an animal of a different species.

As used herein, the term “chimeric antigen receptor” or “CAR,” refers to an artificial receptor that is engineered to be expressed on an immune cell and specifically bind a target protein or antigen. CARs may be used as a therapy with adoptive cell transfer, in which T cells are removed from a patient and modified so that they express a CAR and are then re-introduced into the patient. In some embodiments, the CARs have specificity to a selected target, e.g., cells expressing a prostate-specific membrane antigen. CARs may also comprise an intracellular activation domain, a transmembrane domain and an extracellular domain comprising a tumor associated antigen binding region.

As used herein, the phrase “co-stimulatory ligand,” includes a molecule on an antigen presenting cell (e.g., an artificial APC (aAPC), dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, 0X40, CD30, CD40, PD-1, ICOS, lymphocyte function-associated antigen-1 (LFA-1), CD2, CD7, LIGHT, NKG2C, B7-H3, and a ligand that specifically binds with CD83.

As used herein, a “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

As used herein, a “co-stimulatory signal”, refers to a signal, which in combination with a primary signal, such as TCR/CD3 ligation, leads to T cell proliferation and/or upregulation or downregulation of key molecules.

As used herein, the phrases “effective amount” and “therapeutically effective amount” are used interchangeably, and refer to an amount of a compound, formulation, material, or composition, as described herein effective to achieve a particular biological result or provides a therapeutic or prophylactic benefit. Such results may include, but are not limited to an amount that when administered to a mammal (e.g., a human patient), causes a decrease in tumor size, number, or volume; cancer/tumor cell death; and/or tumor regression. The skilled artisan would understand that the amount of the composition administered herein varies and can be readily determined based on a number of factors such as the cancer being treated, the age and health and physical condition of the mammal being treated, the stage of the cancer/severity of the disease, the particular compound being administered, and the like.

As used herein, the phrase “specifically binds,” with respect to a CAR or the antigen-binding domain thereof, means the CAR recognizes a specific antigen, but does not substantially recognize or bind other molecules in a sample. For example, a CAR that specifically binds to an antigen from one species may also bind to that antigen from one or more species. But, such cross-species reactivity does not itself alter the classification of an antibody as specific. In another example, a CAR that specifically binds to an antigen may also bind to different allelic forms of the antigen. However, such cross reactivity does not itself alter the classification as specific.

### II. Chimeric Antigen Receptors

In some aspects, the present disclosure provides compositions and methods for modified immune cells or precursors thereof, e.g., T cells or natural killer cell (NK cells), comprising a chimeric antigen receptor (CAR). Thus, in some aspects, the immune cell has been genetically modified to express the CAR. In some aspects, CARs of the present disclosure comprise an antigen binding domain, a transmembrane domain, a hinge domain, and an intracellular signaling domain. In some aspects, the CARs comprise an antigen binding domain, a spacer domain, a transmembrane domain, a costimulatory domain, and a signaling domain.

In some aspects, the antigen binding domain may be operably linked to another domain of the CAR, such as the transmembrane domain and/or the intracellular domain, both described elsewhere herein, for expression in the cell. In some aspects, a first nucleic acid sequence encoding the antigen binding domain is operably linked to a second nucleic acid encoding a transmembrane domain, and further operably linked to a third a nucleic acid sequence encoding an intracellular domain.

In some aspects, the antigen binding domains described herein can be combined with any of the transmembrane domains described herein, any of the intracellular domains or cytoplasmic domains described herein, or any of the other domains described herein that may be included in a CAR of the present invention. In some aspects, the CAR may also include a spacer domain as described herein. In some aspects, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In some aspects, the CAR comprises affinity to a target antigen on a target cell. The target antigen may include any type of protein, or epitope thereof, associated with the target cell. For example, the CAR may comprise affinity to a target antigen on a target cell that indicates a particular disease state of the target cell.

In an exemplary aspect, the target cell antigen is an IL-13Rα2 expressed on the cell surface. In some aspects, the CAR has affinity or specificity for IL-13Rα2, an IL-13Rα2 epitope, an IL-13Rα2 mutant, and/or an IL-13Rα2 fragment.

In another exemplary aspect, the target cell antigen is HER2 expressed on the cell surface. In some aspects, the CAR has affinity or specificity for HER2, a HER2 epitope, a HER2 mutant, and/or a HER2 fragment.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. In an exemplary embodiment, a CAR of the present disclosure having affinity for IL-13Rα2 or HER2 on a target cell may comprise an IL-13Rα2- or HER2-binding domain. In some embodiments, the binding domain is a murine binding domain, e.g., the binding domain is of murine origin. In some embodiments, the binding domain is a human binding domain, e.g., the IL-binding domain is of human origin.

In some aspects, a CAR of the present disclosure may have affinity for one or more target antigens on one or more target cells. In some aspects, a CAR may have affinity for one or more target antigens on a target cell. In such aspects, the CAR is a bispecific CAR, or a multispecific CAR. In some aspects, the CAR comprises one or more target-specific binding domains that confer affinity for one or more target antigens. In some aspects, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

In some aspects, the antigen binding domain is selected from the group consisting of an antibody, an antigen binding fragment (Fab), and a single-chain variable fragment (scFv). In some embodiments, an IL-13Rα2 or HER2 binding domain of the present invention is selected from the group consisting of an IL-13Rα2- or HER2-specific antibody, an IL-13Rα2- or HER2-specific Fab, and an IL-13Rα2- or HER2-specific scFv. In one embodiment, an IL-13Rα2 or HER2-binding domain is an IL-13Rα2- or HER2-specific antibody. In one embodiment, an IL-13Rα2- or Her2-binding domain is an IL-13Rα2- or HER2-specific Fab. In one embodiment, an IL-13Rα2- or HER2-binding domain is an IL-13Rα2- or HER2-specific scFv. In some aspects the antigen binding domain is an IL-13 cytokine or a mutant, variant, or fragment thereof (i.e., a “zetakine”). For example, in some embodiments, the antigen binding domain may comprise an IL13 E13Y mutein.

The antigen binding domain can include any domain that binds to the antigen and may include, but is not limited to, an IL-13 protein, polypeptide, variant, mutant, or fragment thereof that is capable of binding to IL-13Rα2 or a protein or polypeptide capable of binding to HER2; a monoclonal antibody; a polyclonal antibody; a synthetic antibody; a human antibody; a humanized antibody; a non-human antibody; and any fragment or scFv thereof. In some embodiments, the antigen binding domain portion comprises a mammalian antibody or a fragment thereof. The choice of antigen binding domain may depend upon the type and number of antigens that are present on the surface of a target cell. As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., IL-13Rα2 binding domain) comprises an scFv having the configuration from N-terminus to C-terminus, VH-linker-VL. In some embodiments, the antigen binding domain (e.g., IL-13Rα2 binding domain) comprises an scFv having the configuration from N-terminus to C-terminus, VL-linker-VH. Those of skill in the art would be able to select the appropriate configuration for use in the present invention. The linker of an scFv is usually rich in glycine for flexibility, as well as serine or threonine for solubility. The linker can link the heavy chain variable region and the light chain variable region of the extracellular antigen-binding domain. Non-limiting examples of linkers are disclosed in WO 2014/087010.

In some aspects, the antigen binding domain comprises

(SEQ ID NO: 1)
GPVPPSTALRYLIEELVNITQNQKAPLCNGSMVWSINLTAGMYCAALESL
INVSGCSAIEKTQRMLSGFCPHKVSAGQFSSLHVRDTKIEVAQFVKDLLL
HLKKLFREGRFN.

In some aspects, the antigen binding domain comprises

(SEQ ID NO: 9)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGSTSGGGSGGGSGGGGSSEVQLVESGGGLVQPGGSLRLSCAAS
GFNIKDTYIHWVRQAPGKGLEWVARIYPTNGYTRYADSVKGRFTISADTS
KNTAYLQMNSLRAEDTAVYYCSRWGGDGFYAMDYWGQGTLVTVSS

In some aspects, tolerable variations in the binding domain will be known to those of skill in the art, while maintaining binding to IL-13Rα2. In some aspects, the IL-13Rα2 binding domain comprises an amino acid sequence that has at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to SEQ ID NO:1 or SEQ ID NO: 9.

### Spacer Domain

In some aspects, the CAR comprises a spacer domain. In some aspects, the spacer domain is an oligopeptide or polypeptide that functions to link one or more of the antigen binding domain, transmembrane domain, costimulatory domain, and signaling domain to one or more of the antigen binding domain, transmembrane domain, costimulatory domain, and signaling domain. In some aspects, the spacer domain may be a short amino acid linker comprising 2, 3, 4, 5, 6, 7, 8, 9 or 10 amino acids in length. For example, a glycine-serine doublet. In some aspects, the spacer domain occurs between the intracellular domain and the transmembrane domain of the CAR. In some aspects the spacer domain occurs between the extracellular domain and the transmembrane domain. In some aspects, the spacer domain may comprise up to 300 amino acids, e.g., 10 to 100 amino acids, or 25 to 50 amino acids.

Non-limiting examples of linkers are disclosed in WO 2015/105522.

In some aspects, the spacer domain comprises an immunoglobulin Fc domain. In some aspects, the spacer domain comprises an IgG Fc domain. In some aspects, the spacer domain comprises an IgG4 Fc domain. In some aspects, the IgG4 Fc domain comprises one of the following:

(SEQ ID NO: 10)
ESKYGPPCPPCPAPEFEGGPSVFLEPPKPKDTLMISRTPEVTCVVVDVSQ
EDPEVQFNWYVDGVEVHNAKTKPREEQFQSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK
(SEQ ID NO: 2)
ESKYGPPCPPCPGGGSSGGGSGGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK;
(SEQ ID NO: 3)
ESKYGPPCPSCPAPEFEGGPSVFLFPPKPKDTLMISRTPEVTCVVVDVSQ
EDPEVQFNWYVDGVEVHQAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK;
(SEQ ID NO: 4)
PKDTLMISRTPEVTCVVVDVSQEDPEVQFNWYVDGVEVHQAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLG
K;
or
(SEQ ID NO: 5)
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK.

In some aspects, tolerable variations in the IgG4 Fc domain will be known to those of skill in the art. In some aspects, the IgG4 Fc domain comprises an amino acid sequence that has at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs:2-5 or 10.

### Hinge Domain

In some aspects, the CAR comprises a hinge domain. The hinge domain of the CAR is a hydrophilic region which can be located between the antigen binding domain and the transmembrane domain. In some aspects, this domain may facilitate proper protein folding for the CAR, among other functions. The hinge domain is an optional component for the CAR. In some aspects, the transmembrane domain further comprises a hinge domain. The hinge domain may include a domain selected from Fc fragments of antibodies, hinge regions of antibodies, CH2 regions of antibodies, CH3 regions of antibodies, artificial hinge sequences or combinations thereof. Examples of hinge domains include, without limitation, a CD8a hinge, artificial hinges made of polypeptides which may be as small as, three glycines (Gly), as well as CH1 and CH3 domains of IgGs (such as human IgG4).

In some aspects, the CAR includes a hinge domain that connects the antigen binding domain with the transmembrane domain, which, in turn, connects to the intracellular domain. The hinge domain is preferably capable of supporting the antigen binding domain to recognize and bind to the target antigen on the target cells. In some aspects, the hinge domain is a flexible domain, thus allowing the antigen binding domain to have a structure to optimally recognize the specific structure and density of the target antigens on a cell such as tumor cell. The flexibility of the hinge domain permits the hinge region to adopt many different conformations.

In some embodiments, the hinge domain is an immunoglobulin heavy chain hinge region. In some embodiments, the hinge domain is a polypeptide derived from a receptor (e.g., a CD8-derived hinge region).

In some aspects, the hinge domain can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

In some aspects, the hinge domain can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

### Transmembrane Domain

In some aspects, CARs of the present disclosure may comprise a transmembrane domain that connects the antigen binding domain of the CAR to the intracellular domain of the CAR. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some aspects, the transmembrane domain is interposed between the antigen binding domain and the intracellular domain of a CAR.

In some aspects, the transmembrane domain is naturally associated with one or more of the domains in the CAR. In some aspects, the transmembrane domain can be selected or modified by one or more amino acid substitutions to avoid binding of such domains to the transmembrane domains of the same or different surface membrane proteins, to minimize interactions with other members of the receptor complex. The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. In some aspects, the transmembrane domain is a transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some aspects, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some aspects, the transmembrane domain comprises a CD4 transmembrane domain. In some aspects, the CD4 transmembrane domain comprises MALIVLGGVAGLLLFIGLGIFF (SEQ ID NO: 6). In some aspects, the transmembrane domain comprises a CD8 transmembrane domain. In some aspects, the CD8 transmembrane domain comprises

(SEQ ID NO: 11)
IYIWAPLAGTCGVLLLSLVIT,
(SEQ ID NO: 12)
IYIWAPLAGTCGVLLLSLVITLY,
or
(SEQ ID NO: 13)
IYIWAPLAGTCGVLLLSLVITLYC

In some aspects, tolerable variations in the transmembrane domain will be known to those of skill in the art. In some aspects, the transmembrane domain comprises an amino acid sequence that has at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to SEQ ID NO:6 or SEQ ID NO: 13.

### Costimulatory Domain

In some aspects, costimulatory signals are necessary to achieve robust CAR-T cell expansion, function, persistence, and anti-tumor activity. These can be provided by incorporating one or more costimulatory domains from one or more T cell costimulatory molecules. In some aspects, costimulatory domains are selected from the costimulatory molecules of CD3, CD4, CD8, T cell receptor (TCR), CD27, CD28, 4-1BB (CD137), 0X40, CD30, CD40, PD-1, ICOS, lymphocyte function-associated antigen-1 (LFA-1), CD2, CD7, LIGHT, NKG2C, B7-H3, a ligand that specifically binds with CD83, or any fragment thereof.

In some aspects, the costimulatory domain comprises a 4-1BB costimulatory domain. In some aspects, the 4-1BB costimulatory domain comprises

(SEQ ID NO: 7)
KRGRKKLLYIFKQPFMRPVQTTQEEDGCSCRFPEEEEGGCEL

In some aspects, tolerable variations in the costimulatory domain will be known to those of skill in the art. In some aspects, the costimulatory domain comprises an amino acid sequence that has at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to SEQ ID NO:7.

### Intracellular Signaling Domain

In some aspects, the CAR also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

In some aspects, the intracellular signaling domain is the cytoplasmic portion of a surface receptor, co-stimulatory molecule, or any molecule that acts in concert to initiate signal transduction in the T cell, as well as any derivative or variant of these elements and any synthetic sequence that has the same functional capability.

In some aspects, the intracellular signaling domain is the z chain of the T cell receptor complex or any of its homologs, e.g., h chain, FcsRfy and b chains, MB 1 (IgA) chain, B29 (Ig) chain, etc., human CD3 ζ chain, CD3 polypeptides (A, d and e), syk family tyrosine kinases (Syk, ZAP 70, etc.), src family tyrosine kinases (Lck, Fyn, Lyn, etc.), and other molecules involved in T cell transduction, such as CD2, CD5 and CD28. In some aspects, the intracellular signaling domain may be human CD3 ζ chain, FcRyIII, FcsRI, cytoplasmic tails of Fc receptors, an immunoreceptor tyrosine-based activation motif (IT AM) bearing cytoplasmic receptors, and combinations thereof.

In some aspects the intracellular signaling domain includes a fragment or domain from one or more molecules or receptors including, but not limited to, TCR, CD3 zeta, CD3 gamma, CD3 delta, CD3 epsilon, CD86, common FcR gamma, FcR beta (Fc Epsilon Rib), CD79a, CD79b, Fcgamma Rlla, DAP 10, DAP 12, T cell receptor (TCR), CD8, CD27, CD28, 4-1BB (CD137), OX9, 0X40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD 160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1 Id, ITGAE, CD 103, ITGAL, CD 11 a, LFA-1, ITGAM, CD lib, ITGAX, CD 11c, ITGB1, CD29, ITGB2, CD 18, LFA-1, ITGB7, TNFR2, TRANCE/RANKL, DNAM1 (CD226), SLAMF4 (CD244, 2B4), CD84, CD 96 (Tactile), CEACAM1, CRT AM, Ly9 (CD229), CD160 (BY55), PSGL1, CD 100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co stimulatory molecule that has the same functional capability, and any combination thereof.

In some aspects, the intracellular signaling domain is a CD3 signaling domain, such as

(SEQ ID NO: 15)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In some aspects, the CAR of the present disclosure comprises or consists of

(SEQ ID NO: 8)
GPVPPSTALRYLIEELVNITQNQKAPLCNGSMVWSINLTAGMYCAALESL
INVSGCSAIEKTQRMLSGFCPHKVSAGQESSLHVRDTKIEVAQFVKDLLL
HLKKLFREGRFNESKYGPPCPPCPAPEFEGGPSVFLFPPKPKDTLMISRT
PEVTCVVVDVSQEDPEVQFNWYVDGVEVHNAKTKPREEQFQSTYRVVSVL
TVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFL
YSRLTVDKSRWQEGNVESCSVMHEALHNHYTQKSLSLSLGKMALIVLGGV
AGLLLFIGLGIFFKRGRKKLLYIFKQPFMRPVQTTQEEDGCSCRFPEEEE
GGCELGGGRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPR.

In some aspects, the CAR of the present disclosure comprises or consists of

(SEQ ID NO: 14)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGSTSGGGSGGGSGGGGSSEVQLVESGGGLVQPGGSLRLSCAAS
GFNIKDTYIHWVRQAPGKGLEWVARIYPTNGYTRYADSVKGRFTISADTS
KNTAYLQMNSLRAEDTAVYYCSRWGGDGFYAMDYWGQGTLVTVSSESKYG
PPCPPCPAPEFEGGPSVFLFPPKPKDTLMISRTPEVTCVVVDVSQEDPEV
QFNWYVDGVEVHNAKTKPREEQFQSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSLGKIYIWAPLAGTCGVLLLSLVITKRGRK
KLLYIFKQPFMRPVQTTQEEDGCSCRFPEEEEGGCELGGGRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR.

In some aspects, tolerable variations in the CAR will be known to those of skill in the art. In some aspects, the CAR comprises an amino acid sequence that has at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to SEQ ID NO:8 or SEQ ID NO: 14.

In some aspects, cells expressing the CAR is introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, or an NK T cell.

When a CAR is expressed in vivo, it may temporarily comprise a leader sequence or GMCSFRa signal peptide, such as MLLLVTSLLLCELPHPAFLLIP (SEQ ID NO: 16), which is ultimately cleaved off of the mature CAR.

The present disclosure also provides genetically engineered cells which include and stably express a CAR or the present disclosure. In some aspects, the engineered cells express a dominant negative receptor and/or switch receptor, and/or bispecific antibody, and/or combinations thereof, of the present disclosure. In some aspects, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In some aspects, the genetically engineered cells are autologous cells. Modified cells (e.g., comprising a subject CAR, dominant negative receptor and/or switch receptor, and/or expresses and secretes a bispecific antibody, and/or combinations thereof) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR, dominant negative receptor and/or switch receptor, and/or bispecific antibody, and/or combinations thereof, of the present disclosure may be expanded ex vivo.

In some aspects, the disclosure is drawn to a population of modified immune cells that express a CAR. In some aspects, the population comprises one or more subpopulation of T cells, NK cells, and/or macrophages.

In some aspects, the population of cells that express the CAR further express a selection tag. In some aspects, the selection tag is a truncated CD19 sequence (CD19t) (e.g., CGDVEENPGPRMPPPRLLFFLLFLTPMEVRPEEPLVVKVEEGDNAVLQCLKGTSDGPTQ QLTWSRESPLKPFLKLSLGLPGLGIHMRPLAIWLFIFNVSQQMGGFYLCQPGPPSEKAWQ PGWTVNVEGSGELFRWNVSDLGGLGCGLKNRSSEGPSSPSGKLMSPKLYVWAKDRPEI WEGEPPCVPPRDSLNQSLSQDLTMAPGSTLWLSCGVPPDSVSRGPLSWTHVHPKGPKSL LSLELKDDRPARDMWVMETGLLLPRATAQDAGKYYCHRGNLTMSFHLEITARPVLWH WLLRTGGWKVSAVTLAYLIFCLCSLVGILHLQRALVLRRKR; SEQ ID NO: 17). In some aspects, the selection tag is a truncated epidermal growth factor receptor sequence (EGFRt). In some aspects, the population of cells that express the CAR comprise a tag that allows for isolation and purification of CAR-expressing cells. Tags such as CD19t and EGFRt may be separated from the mature CAR sequence by a T2A skip sequence (e.g., LEGGGEGRGSLLT; SEQ ID NO: 18).

### III. Oncolytic Viruses

Oncolytic viruses are a form of immunotherapy that utilizes viruses to specifically infect and destroy cancer cells. These viruses, some naturally occurring and some synthetic or modified represent a promising approach to treating cancer for several reasons. Cancer cells often have impaired antiviral defenses that make them susceptible to infection. Naturally occurring oncolytic viruses can be modified to give them advantageous properties, such as decreasing their ability to infect healthy cells. After infection, these viruses can cause cancer cells to lyse, killing the cancer cells and releasing cancer cell antigens. These antigens can then stimulate immune responses that bolster the anti-cancer activity of the immune system.

In some aspects, the oncolytic virus is selected from reovirus, New Castle Disease virus (NDV), vesicular stomatitis virus (VSV), measles virus, influenza virus, Sinbis virus, adenovirus, poxvirus, and herpes virus (HSV). In some aspects, the oncolytic virus is selected from adenoviruses, herpesviruses, poxviruses, picornaviruses, polioviruses, paramyxoviruses, reoviruses, parvoviruses, and rhabdoviruses. In some aspects, the oncolytic virus is a vaccinia virus.

Globally, two viruses, T-VEC and H101 have achieved regulatory approval. H101 is a genetically modified oncolytic adenovirus that was approved in China for the treatment of nasopharyngeal carcinoma in combination with systemic chemotherapy. T-Vec is a recombinant granulocyte macrophage colony-stimulating factor (GM-CSF)-containing human herpes simplex type I virus (HSV-1), for inoperable locally advanced or metastatic malignant melanoma.

For the purposes of the present disclosure, oncolytic herpes simplex viruses are preferred for combining with the disclosed anti-IL-13Rα2 CAR. Oncolytic herpes simplex virus (oHSV) type I constructs are anti-neoplastic agents for a variety of malignancies, including malignant glioma. Clinical trials to date have supported the safety of these agents even when directly administered in the CNS. Traditional pre-clinical US Food and Drug Administration (FDA) toxicity studies for these agents have included the use of two species, generally including murine and primate studies. In particular, C134, a chimeric oHSV construct, have been shown to be safe and effective in a number of model systems. C134 is a neurovirulent, oncolytic second-generation, replication-competent, recombinant and genetically-engineered herpes simplex virus type 1 (HSV-1) where the gene for ICP34.5 has been deleted and the gene encoding the human cytomegalovirus (HCMV), protein kinase R (PKR) evasion protein IRS1, with potential oncolytic and immunostimulating activities. Upon intratumoral administration, oncolytic HSV-1 C134 specifically infects and replicates within the rapidly dividing, glioma cells, thereby directly lysing tumor cells. The released virus particles, in turn, infect and replicate in neighboring tumor cells, thereby further killing tumor cells. Tumor antigens released from the lysed tumor cells also activate the immune system to induce a tumor-specific systemic immune and cytotoxic T-lymphocyte (CTL) response, thereby killing nearby non-infected tumor cells. Deletion of the gene encoding for ICP34.5 imparts tumor selectivity by preventing replication in healthy cells. IRS1 expression allows the virus to replicate within tumors but limits viral spread.

In some aspects, the oncolytic virus is genetically modified. In some aspects, one or more genes in the virus are disrupted. In some aspects, one or more genes in the virus are deleted. In some aspects, one or more genes heterologous to the virus are introduced into the virus. In some aspects, the genetically modified virus is HSV-1. In some aspects, the HSV-1 is modified such that a ICP34.5 gene is disrupted or deleted. In some aspects, the HSV-1 is modified such that a human cytomegalovirus (HCMV) IRS1 gene is introduced into the viral genome. In some aspects, the HSV-1 virus is C134. In some aspects, the oncolytic virus is replication-competent.

### IV. Pharmaceutical Compositions

Pharmaceutical compositions of the present invention may comprise a genetically modified immune cell as described herein, in combination with one or more pharmaceutically or physiologically acceptably carriers, diluents, adjuvants, or excipients. Such compositions may comprise buffers such as neutral buffered saline, phosphate buffered saline and the like; carbohydrates such as glucose, mannose, sucrose, or dextrans, mannitol; proteins; polypeptides or amino acids such as glycine, antioxidants; chelating agents such as EDTA or glutathione; adjuvants (e.g., aluminum hydroxide); and preservatives. In some aspects, compositions of the present invention are preferably formulated for intravenous administration. In some aspects, the pharmaceutical compositions of the present invention are preferably formulated for administration into the central nervous system. In some aspects, the pharmaceutical compositions comprising the retroviral (e.g., lentiviral) vectors are suitable for administering to mammals, preferably humans.

### V. Methods of Treatment

In some aspects, a patient in need is administered both (1) a population of cells that express a CAR, and (2) an oncolytic virus. In some aspects the patient in need is a patient with cancer. In some aspects, the cancer comprises cells expressing IL-13Rα2 and/or HER2. In some embodiments, the cancer is glioblastoma multiforme (GBM).

In some aspects, the patient in need is a female. In some aspects, the patient in need is a male. In some aspects, the patient is an adult (18+ years of age). In some aspects, the patient is a child. In some aspects, the child's age is greater than 2 years old, greater than 3 years old, greater than 4 years old, greater than 5 years old, greater than 6 years old, greater than 7 years old, greater than 8 years old, greater than 9 years old, greater than 10 years old, greater than 11 years old, greater than 12 years old, greater than 13 years old, greater than 14 years old, greater than 15 years old, or greater than 16 years old. In some aspects the patient is an adult greater than 50 years old, greater than 55 years old, greater than 60 years old, greater than 65 years old, greater than 70 years old, greater than 75 years old, greater than 80 years old, greater than 85 years old, greater than 90 years old, greater than 95 years old, greater than 100 years old, or greater than 105 years old.

In some aspects, the patient has had a recurrence or relapse of cancer after a remission period of at least 6 months, at least 9 months, at least 12 months, at least 18 months, at least 24 months, at least 36 months, at least 48 months, at least 5 years, at least 6 years, at least 7 years, at least 8 years, at least 9 years, or at least 10 years.

In some aspects, the patient has undergone surgery to remove a solid tumor. In some aspects, the solid tumor is any one of the cancers described herein. In some aspects, the solid tumor is a metastatic tumor. In some aspects, the surgery was removal of a primary glioblastoma. In some aspects the glioblastoma is glioblastoma multiforme. In some aspects, the patient's cancer was unresponsive to oncolytic therapy alone. In some aspects, the patient's cancer was unresponsive to CAR-T therapy alone.

In some aspects, the patient is administered the population of cells comprising the CAR and the oncolytic virus concurrently. In some aspects, the patient is administered the population of cells comprising the CAR and the oncolytic virus separately. In some aspects, the patient is administered the population of cells comprising the CAR first and the oncolytic virus is administered second. In some aspects, the patient is administered the oncolytic virus first and the population of cells comprising the population of cells comprising the CAR second.

In some aspects, the patient is administered at least one dose of each the oncolytic virus and the population of cells comprising the CAR. In some aspects, the patient is administered at least two doses of each the oncolytic virus and the population of cells comprising the CAR. In some aspects, the patient is administered at least three doses of each the oncolytic virus and the population of cells comprising the CAR. In some aspects, the patient is administered at least one dose of the oncolytic virus and at least two doses of the population of cells comprising the CAR. In some aspects, the patient is administered at least one dose of the population of cells comprising the CAR and at least two doses of the oncolytic virus.

In some aspects, the population of cells comprising the CAR is administered first, and a period of 1, 2, 3, 4, 5, 6, 7, 8, 9, 10, 11, 12, 13, 14, 15, 16 17, 18, 19, 20, 21, 22, 23, 24, 25, 26, 27, 28, 29, or 30 days passes before the oncolytic virus is administered. In some aspects, the oncolytic virus is administered first, and a period of 1, 2, 3, 4, 5, 6, 7, 8, 9, 10, 11, 12, 13, 14, 15, 16 17, 18, 19, 20, 21, 22, 23, 24, 25, 26, 27, 28, 29, or 30 days passes before the population of cells comprising the CAR is administered.

In some aspects, the population of cells comprising the CAR is administered first, and a period of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 days passes before the oncolytic virus is administered. In some aspects, the oncolytic virus is administered first, and a period of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 days passes before the population of cells comprising the CAR is administered.

In some aspects, the oncolytic virus and the population of cells comprising the CAR are administered via the intraventricular route, via the intratumoral route, via the intrathecal route, into a resected tumor cavity, via the intracranial route, or via directly into the central nervous system.

### VI. Cancers

In some aspects, cancers susceptible to the treatments described herein include cancers in which IL-13Rα2 is expressed. In some aspects, such cancers include solid tumors, such as carcinomas and sarcomas. Carcinomas include malignant neoplasms derived from epithelial cells which infiltrate surrounding tissues which result in metastases. Adenocarcinomas are carcinomas derived from glandular tissue, or from tissues that form recognizable glandular structures. In some aspects, the cancers include sarcomas and fibrosarcomas, which are tumors whose cells are embedded in a fibrillary or homogeneous substance, such as embryonic connective tissue. In some aspects, the cancers include myeloid or lymphoid cancers such as leukemias, lymphomas, and other cancers that typically do not present as a tumor mass, but generally distributed in the vascular or lymphoreticular systems. In some aspects, the cancers include cancers unique or generally unique to adults, children, males, and/or females. In some aspects, the cancers include primary cancers, secondary cancers, pre-metastatic cancers, post-metastatic cancers, acute cancers, chronic cancers, benign cancers, malignant cancers, cancers generally localized to an anatomical location.

In some aspects, carcinomas that may be treated include adrenocortical, acinar, acinic cell, acinous, adenocystic, adenoid cystic, adenoid squamous cell, cancer adenomatosum, adenosquamous, adnexel, cancer of adrenal cortex, adrenocortical, aldosterone-producing, aldosterone-secreting, alveolar, alveolar cell, ameloblastic, ampullary, anaplastic cancer of thyroid gland, apocrine, basal cell, basal cell, alveolar, comedo basal cell, cystic basal cell, morphea-like basal cell, multicentric basal cell, nodulo-ulcerative basal cell, pigmented basal cell, sclerosing basal cell, superficial basal cell, basaloid, basosquamous cell, bile duct, extrahepatic bile duct, intrahepatic bile duct, bronchioalveolar, bronchiolar, bronchioloalveolar, bronchoalveolar, bronchoalveolar cell, bronchogenic, cerebriform, cholangiocellular, chorionic, choroids plexus, clear cell, cloacogenic anal, colloid, comedo, corpus, cancer of corpus uteri, cortisol-producing, cribriform, cylindrical, cylindrical cell, duct, ductal, ductal cancer of the prostate, ductal cancer in situ (DCIS), eccrine, embryonal, cancer en cuirasse, endometrial, cancer of endometrium, endometroid, epidermoid, cancer ex mixed tumor, cancer ex pleomorphic adenoma, exophytic, fibrolamellar, cancer fibrosum, follicular cancer of thyroid gland, gastric, gelatinform, gelatinous, giant cell, giant cell cancer of thyroid gland, cancer gigantocellulare, glandular, granulose cell, hepatocellular, Hurthle cell, hypernephroid, infantile embryonal, islet cell carcinoma, inflammatory cancer of the breast, cancer in situ, intraductal, intraepidermal, intraepithelial, juvenile embryonal, Kulchitsky-cell, large cell, leptomeningeal, lobular, infiltrating lobular, invasive lobular, lobular cancer in situ (LCIS), lymphoepithelial, cancer medullare, medullary, medullary cancer of thyroid gland, medullary thyroid, melanotic, meningeal, Merkel cell, metatypical cell, micropapillary, cancer molle, mucinous, cancer muciparum, cancer mucocellulare, mucoepidermoid, cancer mucosum, mucous, nasopharyngeal, neuroendocrine cancer of the skin, noninfiltrating, non-small cell, non-small cell lung cancer (NSCLC), oat cell, cancer ossificans, osteoid, Paget's disease of the bone or breast, papillary, papillary cancer of thyroid gland, periampullary, preinvasive, prickle cell, primary intrasseous, renal cell, scar, schistosomal bladder, Schneiderian, scirrhous, sebaceous, signet-ring cell, cancer simplex, small cell, small cell lung cancer (SCLC), spindle cell, cancer spongiosum, squamous, squamous cell, terminal duct, anaplastic thyroid, follicular thyroid, medullary thyroid, papillary thyroid, trabecular cancer of the skin, transitional cell, tubular, undifferentiated cancer of thyroid gland, uterine corpus, verrucous, villous, cancer villosum, yolk sac, squamous cell particularly of the head and neck, esophageal squamous cell, and oral cancers and carcinomas.

In some aspects, sarcomas that may be treated include adipose, alveolar soft part, ameloblastic, avian, botryoid, sarcoma botryoides, chicken, chloromatous, chondroblastic, clear cell sarcoma of kidney, embryonal, endometrial stromal, epithelioid, Ewing's, fascial, fibroblastic, fowl, giant cell, granulocytic, hemangioendothelial, Hodgkin's, idiopathic multiple pigmented hemorrhagic, immunoblastic sarcoma of B cells, immunoblastic sarcoma of T cells, Jensen's, Kaposi's, Kupffer cell, leukocytic, lymphatic, melanotic, mixed cell, multiple, lymphangio, idiopathic hemorrhagic, multipotential primary sarcoma of bone, osteoblastic, osteogenic, parosteal, polymorphous, pseudo-Kaposi, reticulum cell, reticulum cell sarcoma of the brain, rhabdomyosarcoma, Rous, soft tissue, spindle cell, synovial, telangiectatic, sarcoma (osteosarcoma)/malignant fibrous histiocytoma of bone, and soft tissue sarcomas.

In some aspects, lymphomas that may be treated include AIDS-related, non-Hodgkin's, Hodgkin's, T-cell, T-cell leukemia/lymphoma, African, B-cell, B-cell monocytoid, bovine malignant, Burkitt's, centrocytic, lymphoma cutis, diffuse, diffuse, large cell, diffuse, mixed small and large cell, diffuse, small cleaved cell, follicular, follicular center cell, follicular, mixed small cleaved and large cell, follicular, predominantly large cell, follicular, predominantly small cleaved cell, giant follicle, giant follicular, granulomatous, histiocytic, large cell, immunoblastic, large cleaved cell, large noncleaved cell, Lennert's, lymphoblastic, lymphocytic, intermediate; lymphocytic, intermediately differentiated, plasmacytoid; poorly differentiated lymphocytic, small lymphocytic, well differentiated lymphocytic, lymphoma of cattle; MALT, mantle cell, mantle zone, marginal zone, Mediterranean lymphoma mixed lymphocytic-histiocytic, nodular, plasmacytoid, pleomorphic, primary central nervous system, primary effusion, small b-cell, small cleaved cell, small noncleaved cell, T-cell lymphomas; convoluted T-cell, cutaneous t-cell, small lymphocytic T-cell, undefined lymphoma, u-cell, undifferentiated, aids-related, central nervous system, cutaneous T-cell, effusion (body cavity-based), thymic lymphoma, and cutaneous T cell lymphomas.

In some aspects, leukemias and other blood cell malignancies that may be treated include acute lymphoblastic, acute myeloid, lymphocytic, chronic myelogenous, hairy cell, lymphoblastic, myeloid, lymphocytic, myelogenous, leukemia, hairy cell, T-cell, monocytic, myeloblastic, granulocytic, gross, hand mirror-cell, basophilic, hemoblastic, histiocytic, leukopenic, lymphatic, Schilling's, stem cell, myelomonocytic, prolymphocytic, micromyeloblastic, megakaryoblastic, megakaryocytic, Rieder cell, bovine, aleukemic, mast cell, myelocytic, plasma cell, subleukemic, multiple myeloma, nonlymphocytic, and chronic myelocytic leukemias.

In some aspects, brain and central nervous system (CNS) cancers and tumors that may be treated include astrocytomas (including cerebellar and cerebral), gliomas (including malignant gliomas, glioblastomas, brain stem gliomas, visual pathway and hypothalamic gliomas), brain tumors, ependymoma, medulloblastoma, supratentorial primitive neuroectodermal tumors, primary central nervous system lymphoma, extracranial germ cell tumor, myelodysplastic syndromes, oligodendroglioma, myelodysplastic/myeloproliferative diseases, myelogenous leukemia, myeloid leukemia, multiple myeloma, myeloproliferative disorders, neuroblastoma, plasma cell neoplasm/multiple myeloma, central nervous system lymphoma, intrinsic brain tumors, astrocytic brain tumors, and metastatic tumor cell invasion in the central nervous system.

In some aspects, gastrointestinal cancers that may be treated include extrahepatic bile duct cancer, colon cancer, colon and rectum cancer, colorectal cancer, gallbladder cancer, gastric (stomach) cancer, gastrointestinal carcinoid tumor, gastrointestinal carcinoid tumors, gastrointestinal stromal tumors, bladder cancers, islet cell carcinoma (endocrine pancreas), pancreatic cancer, islet cell pancreatic cancer, prostate cancer rectal cancer, salivary gland cancer, small intestine cancer, colon cancer, and polyps associated with colorectal neoplasia.

In some aspects, bone cancers that may be treated include osteosarcoma and malignant fibrous histiocytomas, bone marrow cancers, bone metastases, osteosarcoma/malignant fibrous histiocytoma of bone, and osteomas and osteosarcomas.

In some aspects, breast cancers that may be treated include small cell carcinoma and ductal carcinoma.

In some aspects, lung and respiratory cancers that may be treated include bronchial adenomas/carcinoids, esophagus cancer esophageal cancer, esophageal cancer, hypopharyngeal cancer, laryngeal cancer, hypopharyngeal cancer, lung carcinoid tumor, non-small cell lung cancer, small cell lung cancer, small cell carcinoma of the lungs, mesothelioma, nasal cavity and paranasal sinus cancer, nasopharyngeal cancer, nasopharyngeal cancer, oral cancer, oral cavity and lip cancer, oropharyngeal cancer; paranasal sinus and nasal cavity cancer, and pleuropulmonary blastoma.

In some aspects, urinary and reproductive tract cancers that may be treated include cervical cancer, endometrial cancer, ovarian epithelial cancer, extragonadal germ cell tumor, extracranial germ cell tumor, extragonadal germ cell tumor, ovarian germ cell tumor, gestational trophoblastic tumor, spleen, kidney cancer, ovarian cancer, ovarian epithelial cancer, ovarian germ cell tumor, ovarian low malignant potential tumor, penile cancer, renal cell cancer (including carcinomas), renal cell cancer, renal pelvis and ureter (transitional cell cancer), transitional cell cancer of the renal pelvis, and ureter, gestational trophoblastic tumor, testicular cancer, ureter and renal pelvis, transitional cell cancer, urethral cancer, endometrial uterine cancer, uterine sarcoma, vaginal cancer, vulvar cancer, ovarian carcinoma, primary peritoneal epithelial neoplasms, cervical carcinoma, uterine cancer and solid tumors in the ovarian follicle), superficial bladder tumors, invasive transitional cell carcinoma of the bladder, and muscle-invasive bladder cancer.

In some aspects, skin cancers and melanomas that may be treated include cutaneous t-cell lymphoma, intraocular melanoma, tumor progression of human skin keratinocytes, basal cell carcinoma, and squamous cell cancer. Liver cancers that may be targeted include extrahepatic bile duct cancer, and hepatocellular cancers. Eye cancers that may be targeted include intraocular melanoma, retinoblastoma, and intraocular melanoma Hormonal cancers that may be targeted include: parathyroid cancer, pineal and supratentorial primitive neuroectodermal tumors, pituitary tumor, thymoma and thymic carcinoma, thymoma, thymus cancer, thyroid cancer, cancer of the adrenal cortex, and ACTH-producing tumors.

In some aspects, other cancers that may be treated include advanced cancers, AIDS-related, anal cancer, adrenal, cortical, aplastic anemia, aniline, betel or buyo cheek, cerebriform, chimney-sweeps, clay pipe, colloid, contact, cystic, dendritic, cancer a deux, duct, dye workers, encephaloid, cancer en cuirasse, endometrial, endothelial, epithelial, glandular, cancer in situ, kang, kangri, latent, medullary, melanotic, mule-spinners', non-small cell lung, occult cancer, paraffin, pitch workers', scar, schistosomal bladder, scirrhous, lymph node, small cell lung, soft, soot, spindle cell, swamp, tar, tubular cancers, carcinoid (gastrointestinal and bronchial) Castleman's disease chronic myeloproliferative disorders, clear cell sarcoma of tendon sheaths, Ewing's family of tumors, head and neck cancer, lip and oral cavity cancer, Waldenstrom's macroglobulinemia, metastatic squamous neck cancer with occult primary, multiple endocrine neoplasia syndrome, multiple myeloma/plasma cell neoplasm, Wilms' tumor, mycosis fungoides, pheochromocytoma, sezary syndrome, supratentorial primitive neuroectodermal tumors, unknown primary site, peritoneal effusion, malignant pleural effusion, trophoblastic neo-plasms, and hemangiopericytoma.

In some aspects, the cancer that may be treated is any one of the preceding cancers in which IL-13Rα2 is expressed. In some aspects, the IL-13Rα2 is expressed on the extracellular surface of the cancer cells. In some aspects, the cancer is glioblastoma multiforme.

In some aspects, the cancer that may be treated is any one of the preceding cancers in which HER2 is expressed. In some aspects, the HER2 is expressed on the extracellular surface of the cancer cells. In some aspects, the cancer is glioblastoma multiforme.

In addition to cancers, the presently disclosed compositions, uses, and methods can treat leptomeningeal disease. Leptomeningeal disease (LMD; also referred to as leptomeningeal metastases or carcinomatous meningitis) is a rare but frequently devastating complication of advanced cancer from solid tumors, most commonly lung cancer, breast cancer, and melanoma (among others, such as brain cancers). Patients can present with a broad range of signs and symptoms due to simultaneous involvement of multiple areas of the craniospinal axis. Diagnosis often requires a high index of suspicion and is confirmed by neuroimaging and cerebrospinal fluid (CSF) analysis. Patients with LMD have complex needs and are at risk for rapid deterioration. Patients often require multidisciplinary care to achieve optimal symptom control and quality of life. In addition to multifocal neurologic signs and symptoms, patients may have hydrocephalus, seizures, and dysfunction of the hypothalamic-pituitary axis.

Thus, in a further aspect, the present disclosure provides treatments for LMD comprising administering to a subject with GBM (i) a population of cells that express a chimeric antigen receptor (CAR), and (ii) an oncolytic virus. In some embodiments, the population of cells that express the CAR and the oncolytic virus are administered concurrently. In some embodiments, the population of cells that express the CAR and the oncolytic virus are administered sequentially. In some embodiments, the population of cells that express the CAR are administered first and the oncolytic virus is administered second. In some embodiments, the oncolytic virus is administered first and the population of cells that express the CAR are administered second. In some embodiments, the subject has previously undergone surgery to remove a primary GBM tumor and/or a metastatic tumor. In some embodiments, the population of cells that express that CAR comprise T cells, such as central memory T cells. In some embodiments, the population of cells that express that CAR comprise NK cells. In some embodiments, the CAR comprises a binding domain, a spacer domain, a transmembrane domain, a costimulatory domain, and a CD3ζ signaling domain. In some embodiments, the CAR may selectively bind to IL-13Rα2, e.g., the binding domain may comprise SEQ ID NO:1. In some embodiments, the spacer domain comprises an IgG4 Fc domain (e.g., an IgG4 Fc domain comprising SEQ ID NO:2, SEQ ID NO:3, SEQ ID NO:4, or SEQ ID NO:5). In some embodiments, the transmembrane domain comprises a CD4 transmembrane domain (e.g., a TM domain comprising MALIVLGGVAGLLLFIGLGIFF (SEQ ID NO: 6)). In some embodiments, the transmembrane domain comprises a CD8 transmembrane domain (e.g., a TM domain comprising IYIWAPLAGTCGVLLLSLVIT (SEQ ID NO: 11), IYIWAPLAGTCGVLLLSLVITLY (SEQ ID NO: 12), or IYIWAPLAGTCGVLLLSLVITLYC (SEQ ID NO: 13)). In some embodiments, the costimulatory domain comprises a 4-1BB costimulatory domain (e.g., a costimulatory domain comprising SEQ ID NO:7). In some embodiments, the CAR comprises SEQ ID NO: 8 or SEQ ID NO: 14. In some embodiments, the oncolytic virus is a genetically engineered herpes simplex virus type 1 (HSV-1). In some embodiments, the oncolytic virus is replication-competent. In some embodiments, a ICP34.5 gene is deleted from the genome of the oncolytic virus and a gene encoding human cytomegalovirus (HCMV) IRS1 gene is introduced into the genome of the oncolytic virus. In some embodiments, the oncolytic virus is C134. In some embodiments, the population of cells that express the CAR are central memory T cells and the CAR comprises SEQ ID NO: 8 or SEQ ID NO: 14, and the oncolytic virus is C134. In some embodiments, the population of cells expressing the CAR and the oncolytic virus are administered via the same route of administration. In some embodiments, the population of cells expressing the CAR and the oncolytic virus are administered via different routes of administration. In some embodiments, the route of administration is selected from the group consisting of intraventricular, intratumoral, intrathecal, and into a resected tumor cavity. In some embodiments, the population of cells expressing the CAR and the oncolytic virus are administered directly into the central nervous system (CNS) of the subject.

The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular aspects only, and is not intended to be limiting.

### EXAMPLES

### Example 1—Car-T Treatment in U87 Athymic Mouse Model

In some aspects, xenograft models utilizing athymic nude mice are used. Athymic nude mice exhibit abnormal development of the thymus, which leads to severe T-cell dysfunction; however, the mice still have innate immune system components (neutrophils and dendritic cells (DCs), NK cells) and B cells. See Goldman et al. 1998. Br J Haematol, 103(2):335-342. One of the oldest and most widespread animals for creating human xenografts is the athymic nude mice in which human tumor cells are transplanted. This model accurately reflects the complexity mediated by the natural development of the tumor, including genomic heterogeneity, tumor architecture and microenvironment factors, especially if the tumors are transplanted orthotopically. See Wang et al. 2017. Int J Cancer, 140(3):662-673 and Hoffman 2015. Nat Rev Cancer, 15(8):451-452. Xenograft mouse models of hematopoietic and lymphoid tissue tumors are actively used to evaluate and develop new CAR T-cell therapies aimed at various tumors. See Teng et al. 2019. J Immunother, 42(2):33-42 and Zah et al. 2017. Cancer Immunol Res, 4(6):498-508.

U87 is a human primary glioblastoma cell line that is commonly used in brain cancer research. In addition, U87 cell lines were highly permissive to oncolytic HSV infection. See Ghonime et al. 2018. Transl Oncol, 11(1):86-93 and Shah et al. 2007. Gene Ther, 14(13):1045-1054.

A xenograft mouse model that utilizes athymic nude mice is used in which tumors comprising U87 human primary glioblastoma cell line is transplanted into the mice orthotopically. About twenty days passes after transplantation before the CAR-T cells are administered to the mice.

The CAR-Ts of the present disclosure, including those that selectively bind to IL-13Rα2 or HER2, are injected intracranially into the U87 athymic mice at dosages comprising between 0.5×10⁶ to 1.0×10⁶ CAR-T cells for each experiment.

Each experiment comprises three female and three male mice for each sampling time point. The time points are day 0 (D0), 1 (D1), 4 (D4), 8, (D8), and 14 (D14) post-administration of CAR-Ts. For each sampling, mouse brains and blood are collected. The brains are sectioned, and the sections are subjected to immunohistochemistry and immunofluorescence analysis, which utilize antibodies such as anti-CD8, anti-IL-13Rα2 or anti-HER2, anti-PD1, and corresponding secondary antibodies conjugated to various fluorescent molecules.

The persistence of the CAR-Ts are evaluated within the tumors and in the periphery for each time point. Furthermore, the size of the tumors are evaluated relative to the size of the tumors upon implantation.

### Example 2—Oncolytic Virus in U87 Athymic Mouse Model

An oncolytic virus of the present disclosure is administered intracerebrally into U87 athymic mice comprising transplanted tumors comprising U87 human primary glioblastoma cells. About twenty days passes after transplantation before the CAR-T cells are administered to the mice.

Each experiment comprises three female and three male mice for each sampling time point. The time points are day 0 (D0), 1 (D1), 4 (D4), 8, (D8), and 14 (D14) post-tumor implantation. For each sampling, mouse brains and blood are collected. For each time point the mice were evaluated for the recovery of oncolytic virus from the tumors and for the size of the tumors, relative to the size of the tumors upon implantation.

Viral recovery, in vivo, is measured by limiting dilution assay and by TAQMAN quantitative PCR. For the tumor recovery studies, U87-MG tumors were induced in SCID mice and treated with C134 recombinant virus. On day 1, 2, 4, and 8 post-inoculation, three animals from each of the treatment cohorts are sacrificed, their brains recovered, weighed, freeze-thawed, mechanically disrupted by homogenization, and the samples are sonicated prior to quantifying the recovered virus by limiting dilution plaque assay or by TAQMAN quantitative PCR after DNA extraction using a Qiagen biorobot and Qiagen EZ1 tissue kit.

The average recovered virus and standard deviation are calculated for each virus and time point tested. A similar method is used for the in vivo recovery studies from non-malignant CNS. Cohorts of 5-week-old CBA/J mice are injected with equivalent doses (5×10⁵ PFU) of C130 or C101. One days 2, 4, 6, and 8, animals are sacrificed, brains are recovered, and the infectious virus is measured using limiting dilution plaque assays.

### Example 3—Oncolytic Virus-Car-T Combination Treatment in U87 Athymic Mouse Model

In this study, there are four groups of mice: (1) tumor only—labelled, (2) CAR-T only (optimal dose of CAR-T), (3) C134 oncolytic virus only (1×10⁶ pfu), and (4) oncolytic virus C134 followed by CAR-T.

Tumors comprising U87 human primary glioblastoma cells are transplanted into athymic mice. At a later point in time, the mice are injected with (1) CAR-T only, (2) oncolytic virus only, or (3) oncolytic virus followed by CAR-T.

Each experiment comprises three female and three male mice for each group and each sampling time point. The time points are day 0 (D0), 3 (D3), 5, (D5), and 7 (D7) post-administration of the C134 oncolytic virus and/or CAR-T. For the combination C134 oncolytic virus and CAR-T samples, various time points are utilized between the administration of the oncolytic virus and the CAR-T. Each sampling comprises an evaluation of the tumor via live imaging, as compared to the negative control in which no oncolytic virus and/or no CAR-T was administered.

### Example 4—Oncolytic Virus and Car-T Combination Treatment in U87 Athymic Mouse Model for a Period of 3 Months

In this study, there are four groups of mice: (1) tumor only—labelled, (2) CAR-T only, (3) C134 oncolytic virus only (1×10⁶ pfu), and (4) oncolytic virus C134 followed by CAR-T.

Tumors comprising U87 human primary glioblastoma cells are transplanted into athymic mice. At a later point in time, the mice are injected with (1) CAR-T only, (2) oncolytic virus only, or (3) oncolytic virus followed by CAR-T.

Each experiment comprises five female and five male mice for each group and each sampling time point. The time points are day 0 (D0), 3 (D3), 5, (D5), and 7 (D7) post-administration of the C134 oncolytic virus and/or CAR-T. For the combination C134 oncolytic virus and CAR-T samples, various time points are utilized between the administration of the oncolytic virus and the CAR-T.

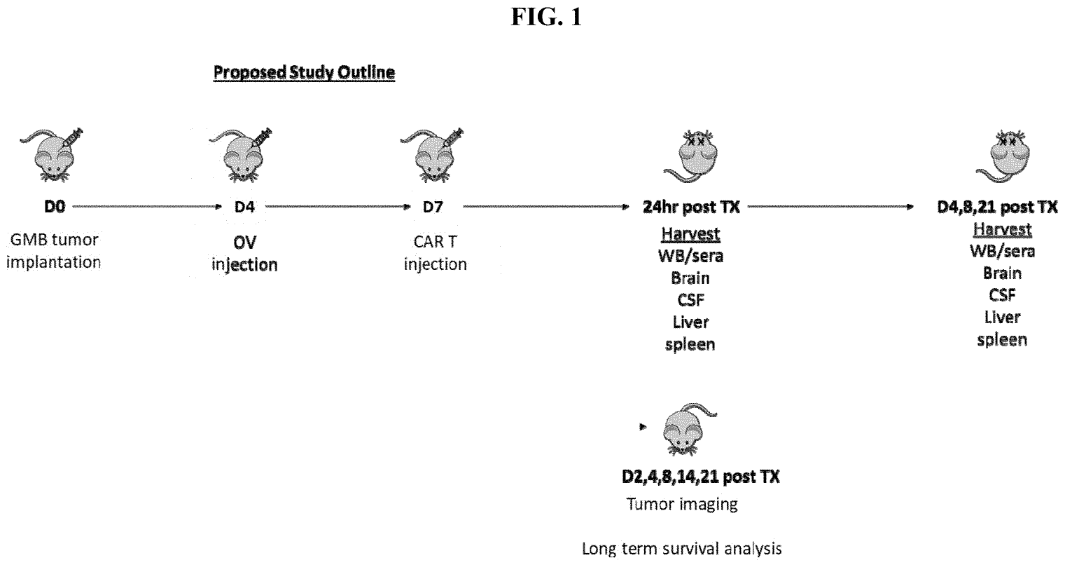

Each sampling comprises an evaluation of (1) CAR-T persistence, (2) C134 persistence, (3) pro-inflammatory cytokines present, (4) the tumor via live imaging, (5) survival, and (6) morbidity. Furthermore, daily clinical observations will be made, which include weight measurements. For each sampling each of the following is harvested post-sacrifice: whole blood/sera, brain, cerebrospinal fluid, liver, and spleen. A graphical representation of the administrations and assays is depicted in FIG. 1. It should be noted that in some embodiments the administration order of the CAR T cells and OV injection may be switched (i.e., CAR T injection on D4 and OV injection on D7).

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred aspects and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular aspects only, and is not intended to be limiting.

All references, articles, publications, patents, patent publications, and patent applications cited herein are incorporated by reference in their entireties for all purposes.

What is claimed:

1. A method of treating glioblastoma multiforme (GBM) in a subject comprising, administering to a subject with GBM (i) a population of cells that express a chimeric antigen receptor (CAR), and (ii) an oncolytic virus.

2. The method of claim 1, wherein the population of cells that express the CAR and the oncolytic virus are administered concurrently.

3. The method of claim 1, wherein the population of cells that express the CAR and the oncolytic virus are administered sequentially.

4. The method of claim 3, wherein the population of cells that express the CAR are administered first and the oncolytic virus is administered second.

5. The method of claim 3, wherein the oncolytic virus is administered first and the population of cells that express the CAR are administered second.

6. The method of claim 1, wherein the subject has previously undergone surgery to remove a primary GBM tumor and/or a metastatic tumor.

7. The method of claim 1, wherein the population of cells that express the CAR comprise T cells.

8. The method of claim 7, wherein the T cells are central memory T cells.

9. The method of claim 1, wherein the population of cells that express the CAR comprise NK cells.

10. The method of claim 1, wherein the CAR comprises a binding domain, a spacer domain, a transmembrane domain, a costimulatory domain, and a CD3ζ signaling domain.

11. The method of claim 1, wherein the CAR selectively binds to IL-13Rα2 or HER2.

12. The method of claim 10, wherein the binding domain comprises
(SEQ ID NO: 1)
GPVPPSTALRYLIEELVNITQNQKAPLCNGSMVWSINLTAGMYCAALESL
INVSGCSAIEKTQRMLSGFCPHKVSAGQFSSLHVRDTKIEVAQFVKDLLL
HLKKLFREGRFN
or
(SEQ ID NO: 9)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGSTSGGGSGGGSGGGGSSEVQLVESGGGLVQPGGSLRLSCAAS
GFNIKDTYIHWVRQAPGKGLEWVARIYPTNGYTRYADSVKGRFTISADTS
KNTAYLQMNSLRAEDTAVYYCSRWGGDGFYAMDYWGQGTLVTVSS.

13. The method of claim 10, wherein the spacer domain comprises an IgG4 Fc domain.

14. The method of claim 13, wherein the IgG4 Fc domain comprises
(i)
(SEQ ID NO: 10)
ESKYGPPCPPCPAPEFEGGPSVFLFPPKPKDTLMISRTPEVTCVVVDVSQ
EDPEVQFNWYVDGVEVHNAKTKPREEQFQSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK;
(ii)
(SEQ ID NO: 2)
ESKYGPPCPPCPGGGSSGGGSGGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK;
(iii)
(SEQ ID NO: 3)
ESKYGPPCPSCPAPEFEGGPSVFLFPPKPKDTLMISRTPEVTCVVVDVSQ
EDPEVQFNWYVDGVEVHQAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK;
(iv)
(SEQ ID NO: 4)
PKDTLMISRTPEVTCVVVDVSQEDPEVQFNWYVDGVEVHQAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLG
K;
or
(v)
(SEQ ID NO: 5)
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK.

15. The method of claim 10, wherein the transmembrane domain comprises a CD4 transmembrane domain or a CD8 transmembrane domain.

16. The method of claim 15, wherein the CD4 transmembrane domain comprises MALIVLGGVAGLLLFIGLGIFF (SEQ ID NO: 6) or wherein the CD8 transmembrane domain comprises IYIWAPLAGTCGVLLLSLVIT (SEQ ID NO: 11), IYIWAPLAGTCGVLLLSLVITLY (SEQ ID NO: 12), or IYIWAPLAGTCGVLLLSLVITLYC (SEQ ID NO: 13).

17. The method of claim 10, wherein the costimulatory domain comprises a 4-1BB costimulatory domain.

18. The method of claim 17, wherein the 4-1BB costimulatory domain comprises
(SEQ ID NO: 7)
KRGRKKLLYIFKQPFMRPVQTTQEEDGCSCRFPEEEEGGCEL.

19. The method of claim 1, wherein the CAR comprises
(SEQ ID NO: 8)
GPVPPSTALRYLIEELVNITQNQKAPLCNGSMVWSINLTAGMYCAALESL
INVSGCSAIEKTQRMLSGFCPHKVSAGQFSSLHVRDTKIEVAQFVKDLLL
HLKKLFREGRFNESKYGPPCPPCPAPEFEGGPSVFLFPPKPKDTLMISRT
PEVTCVVVDVSQEDPEVQFNWYVDGVEVHNAKTKPREEQFQSTYRVVSVL
TVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFL
YSRLTVDKSRWQEGNVFSCSVMHEALHNHYTQKSLSLSLGKMALIVLGGV
AGLLLFIGLGIFFKRGRKKLLYIFKQPFMRPVQTTQEEDGCSCRFPEEEE
GGCELGGGRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPR;
or
(SEQ ID NO: 14)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGSTSGGGSGGGSGGGGSSEVQLVESGGGLVQPGGSLRLSCAAS
GFNIKDTYIHWVRQAPGKGLEWVARIYPTNGYTRYADSVKGRFTISADTS
KNTAYLQMNSLRAEDTAVYYCSRWGGDGFYAMDYWGQGTLVTVSSESKYG
PPCPPCPAPEFEGGPSVFLFPPKPKDTLMISRTPEVTCVVVDVSQEDPEV
QFNWYVDGVEVHNAKTKPREEQFQSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSLGKIYIWAPLAGTCGVLLLSLVITKRGRK
KLLYIFKQPFMRPVQTTQEEDGCSCRFPEEEEGGCELGGGRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR.

20. The method of claim 1, wherein the population of cells that express the CAR also express a selection tag.

21. The method of claim 20, wherein the selection tag is a truncated CD19 sequence (CD19t) or a truncated epidermal growth factor receptor sequence (EGFRt), and, optionally, wherein the CD19t or EGFRt is separated from the CAR by a T2A spacer sequence.

22. The method of claim 1, wherein the oncolytic virus is a genetically engineered herpes simplex virus type 1 (HSV-1).

23. The method of claim 1, wherein the oncolytic virus is replication-competent.

24. The method of claim 22, wherein a ICP34.5 gene is deleted from the genome of the oncolytic virus and a gene encoding human cytomegalovirus (HCMV) IRS1 gene is introduced into the genome of the oncolytic virus.

25. The method of claim 1, wherein the oncolytic virus is C134.

26. The method of claim 1, wherein the population of cells that express the CAR are central memory T cells and the CAR comprises SEQ ID NO: 8 or SEQ ID NO: 14, and wherein the oncolytic virus is C134.

27. The method of claim 1, wherein the population of cells expressing the CAR and the oncolytic virus are administered via the same route of administration.

28. The method of claim 1, wherein the population of cells expressing the CAR and the oncolytic virus are administered via different routes of administration.

29. The method of claim 27, wherein the route of administration is selected from the group consisting of intraventricular, intratumoral, intrathecal, and into a resected tumor cavity.

30. The method of claim 1, wherein the population of cells expressing the CAR and the oncolytic virus are administered directly into the central nervous system (CNS) of the subject.


SEQUENCE LISTING (18 sequences)

SEQ ID NO: 1 - protein, 112 aa, Artificial Sequence
  CAR binding domain
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 2 - protein, 129 aa, Artificial Sequence
  IgG4 Fc spacer domain
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 229 aa, Artificial Sequence
  IgG4 Fc spacer domain
ESKYGPPCPSCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 201 aa, Artificial Sequence
  IgG4 Fc spacer domain
PKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 107 aa, Artificial Sequence
  IgG4 Fc spacer domain
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 22 aa, Artificial Sequence
  CD4 transmembrane domain
MALIVLGGVAGLLLFIGLGIFF

SEQ ID NO: 7 - protein, 42 aa, Artificial Sequence
  4-1BB costimulatory domain
KRGRKKLLYIFKQPFMRPVQTTQEEDGCSCRFPEEEEGGCEL

SEQ ID NO: 8 - protein, 520 aa, Artificial Sequence
  Chimeric antigen receptor (CAR)
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 245 aa, Artificial Sequence
  HER2 CAR Binding Domain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 10 - protein, 229 aa, Homo sapiens
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 21 aa, Homo sapiens
IYIWAPLAGTCGVLLLSLVIT

SEQ ID NO: 12 - protein, 23 aa, Homo sapiens
IYIWAPLAGTCGVLLLSLVITLY

SEQ ID NO: 13 - protein, 24 aa, Homo sapiens
IYIWAPLAGTCGVLLLSLVITLYC

SEQ ID NO: 14 - protein, 652 aa, Artificial Sequence
  HER2-specific Chimeric Antigen Receptor
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 22 aa, Artificial Sequence
  GMCSFRa Signal Peptide Sequence
MLLLVTSLLLCELPHPAFLLIP

SEQ ID NO: 17 - protein, 334 aa, Artificial Sequence
  Modified CD19 Sequence
CGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 18 - protein, 13 aa, Artificial Sequence
  T2A Skip Sequence
LEGGGEGRGSLLT